(19) United States
(12) Patent Application Publication
(10) Pub. No.: US 2007/0248670 A1
(43) Pub. Date: Oct. 25, 2007

(54) TOLTERODINE BEADS

(76) Inventor: Dennie J. M. van den Heuvel, Beuningen (NL)

Correspondence Address: SYNTHON IP INC, 7130 HERITAGE VILLAGE PLAZA, STE 202, GAINESVILLE, VA, 20155, US

(21) Appl. No.: 11/738,248

(22) Filed: Apr. 20, 2007

Related U.S. Application Data
(60) Provisional application No. 60/794,250, filed on Apr. 21, 2006.

(51) Int. Cl. A61K 31/137; A61K 9/24

(52) U.S. Cl. 424/471; 514/649

(57) ABSTRACT

Controlled release tolterodine beads are formed of a sugar core, an innermost sealcoat layer comprising HPMC, a tolterodine drug layer, and an outer control release layer. The thickness of the hydrophilic HPMC sealcoat layer can be used help modulate the release of the tolterodine drug.

This application claims the benefit of priority under 35 U.S.C. § 119(e) from U.S. Provisional Application Ser. No. 60/794,250, filed Apr. 21, 2006, the entire contents of which are incorporated herein by reference.

The present invention relates to pharmaceutical beads having a controlled release of the active substance tolterodine, to a formulation containing said beads and to methods of preparing said beads.

### DESCRIPTION OF THE RELATED ART

Pharmaceutical formulations based on controlled release beads comprising an inert core, such as a sugar sphere, coated with a drug-containing layer and an outer membrane layer for controlling the release rate of the drug are well known in the art. An example of such a controlled release bead can be found in WO96/01621 and its equivalent U.S. Pat. No. 5,783,215. The specifications of these two patents indicate that the presence in certain amounts of a hydrophilic polymer in the drug layer can provide advantageous mechanical properties and may, for the specific drug furosemid, provide for favorable control over dissolution properties below pH 4.

WO00/27364 and corresponding U.S. Pat. No. 6,911,217 relate to controlled release beads containing (i) an inert core, (ii) a water-insoluble polymer layer surrounding the core (iii) a drug layer thereon, and (iv) a controlled release polymer layer. The specification teaches that it was previously “not uncommon” to apply a water-soluble polymer layer between the core and the drug layer, known as a “sealcoat,” to the beads as described in U.S. Pat. No. 5,783,215. Such a water-soluble sealcoat would be present in a small amount, e.g. 1-3%. According to WO00/27364 and U.S. Pat. No. 6,911,217, the purpose of the sealcoat was to isolate the drug from the core surface to prevent any possible chemical interaction and/or to provide a smooth surface on the core with more consistent surface area to thereby obtain improved coating quality and reduced lot-to-lot variations. In contrast, the purported invention in WO00/27364 and U.S. Pat. No. 6,911,217 relates to the use of a sealcoat made of a water-insoluble material that serves to enhance the drug release profile. As seen in FIG. 1 of these patents, the in vitro release of tolterodine becomes slower and more zero-order as the amount of the water-insoluble sealcoat increases from 0% to 14%.

Pharmacia & Upjohn, now Pfizer Inc., markets a prolonged-release capsule formulation of tolterodine tartrate under such brand names as DETROL LA™ in the U.S. and DETRUSITOL SR™ in Europe. Tolterodine is a well known pharmaceutical substance and is useful for treatment of urinary disorders such as overactive bladder. Its chemical name is (R)—N,N-disopropyl-3-(2-hydroxy-5-methylphenyl)-3-phenylpropanamine and it has been disclosed in U.S. Pat. No. 5,382,600. In pharmaceutical applications, tolterodine is typically used in the form of a salt with L-tartaric acid, commonly referred to as tolterodine tartrate. According to the FDA Orange Book, the commercial product sold in the U.S. is covered by U.S. Pat. No. 6,911,217, discussed above. According to this patent, the bead composition is preferably adjusted so that the in vitro dissolution of tolterodine is not more than 30% after 1 hour, from 40 to 85% after 3 hours and not less than 80% after 7 hours.

U.S. Pat. No. 6,630,162 and U.S. Pat. No. 6,770,295 also relate to tolterodine-containing beads and capsule formulations. These patents describe controlled-release formulations, similar to those in U.S. Pat. No. 6,911,217, that obtain certain release profiles or blood plasma levels, respectively. In each of the examples of the invention, the water-insoluble polymer ethylcellulose, under the brand name SURELEASE® (Colorcon, Inc. West Point, Pa., U.S.A.), is used as a sealcoat. The same brand name polymer is also used as the sealcoat in the examples of U.S. Pat. No. 6,911,217.

In a more recent patent application relating to tolterodine-comprising beads, WO 2004-105735 teaches that it is possible to obtain the suitable release profile of tolterodine from coated pellets without the use of the intermediary coating (sealcoat). That is, the tolterodine-containing layer coat is directly coated on the inner core. This possibility was also studied in the U.S. Pat. No. 6,911,217, however it was shown that after two hours the release rate failed to maintain the desired zero order release rate and released the tolterodine too quickly.

It would be desirable to provide an alternative composition of pharmaceutical pellets comprising tolterodine that provided controlled release of the active. In particular, an alternative bead formulation that allows for good scale up and commercial manufacture and which preferably provides a release rate profile similar or equivalent to the marketed controlled release capsules is desirable.

### SUMMARY OF THE INVENTION

The present invention relates to the discovery that a relatively thick water-soluble sealcoat can be successfully employed in tolterodine controlled release beads without losing control of the release profile. Accordingly, a first aspect of the invention relates to a controlled release pharmaceutical bead comprising tolterodine or an acid addition salt thereof as the active substance, such bead comprising:

a) a sugar core of an average diameter 500-1000 microns, preferably 700 to 850 microns, which constitutes 70 to 80% of the total weight of the bead composition;

b) a sealcoat layer comprising or consisting of hydroxypropylmethyl cellulose, which constitutes 5-15% of the total weight of the bead composition, preferably about 5-10%;

c) a drug layer, which constitutes 3-5% of the total weight of the bead composition, comprising tolterodine or an acid addition salt thereof, preferably tolterodine tartrate, and a hydrophilic polymer, preferably hydroxypropylmethylcellulose, such layer preferably containing 50-60% of the tolterodine drug and 40-50% of HPMC, relative to the layer mass; and

d) a control release layer, which constitutes 10-20% of the total weight of the bead composition (preferably about 14%) containing a water insoluble polymer (80-90% of the layer mass) and a pore forming component (10-20%, preferably about 14% of the layer mass).

Furthermore, the above bead can also further comprise an outermost filmcoat, preferably of HPMC, having a weight of about 0.5-2% of the weight of the bead.

In particular, the above bead exhibits the following release rate of tolterodine in a phosphate buffer pH 6.8: not more than 30% (preferably 5-25%) after 1 hour, from 40 to 85% (preferably 45-65%) after 3 hours and not less than 80% after 7 hours.

Another aspect of the invention provides a dosage formulation comprising a plurality of the above controlled-release beads in a capsule.

### BRIEF DESCRIPTION OF DRAWINGS

FIG. 1 shows a comparison of the release rate between the beads from the commercially available tolterodine capsules and the beads of Example 1 of the present invention. The released tolterodine (shown as % TTD) was measured at 37° C. in 800 ml of USP phosphate buffer pH 6.8 on a USP dissolution test apparatus 1 at 100 rpm. The data in FIG. 1 were obtained by an HPLC method and are uncorrected.

### DETAILED DESCRIPTION OF THE INVENTION

Within the present invention, the word “tolterodine” shall also comprise tolterodine salt and particularly the tartrate, if not indicated specifically to the contrary.

The present invention deals with controlled release beads comprising tolterodine. In general, the beads are comprised of a sugar core, an innermost sealcoat layer, a drug layer, and an outer control release layer. Further layers may additionally be present, although typically either no additional layers are present or an optional outermost filmcoat layer is additionally present, if desired. Further details of the bead components are set forth below.

The inner core of the beads consists of or at least comprises a water-soluble sugar. Typically a commercially available sugar sphere, i.e., sucrose/starch spheres (Sugar Spheres NF) is used. Alternatively, similar spheres made from lactose, etc., may be used as the core. Typically, the size of the sphere is within the range of 500-1000 microns, preferably 700 to 850 microns; i.e., the sphere population has an average diameter of 500-1000, preferably 700-850. In weight amounts, the core typically constitutes 70 to 80% of the total weight of the bead composition, and in some embodiments about 75%.

The innermost sealcoat layer comprises hydroxypropyl methyl cellulose (HPMC), a hydrophilic polymer. The HPMC is available in various grades, but generally a low viscosity HPMC is used, typically Pharmacoat 603, in the beads of the invention. The relative weight of the sealcoat is generally from 5 to 15% of the total weight of the bead composition, typically 5-10%, and preferably about 7% in typical embodiments of the invention. The HPMC sealcoat does not serve merely to protect the active substance from contact with the inner core. Instead, the hydrophilic polymer used in the sealcoat layer is desired to cooperate with and/or modify the release profile of the drug in terms of both the rate of penetration of water from the outer environment into the core and the osmotic pressure in the core that helps the release the drug out of the bead. This modulation of release properties is generally achieved by using a thicker than conventional sealcoat layer of HPMC.

The drug layer comprises tolterodine, typically tolterodine tartrate or another water soluble salt of tolterodine, together with a polymer as a binder. Advantageously, the binder is a hydrophilic polymer, and typically it is also an HPMC. Among the commercially available grades, a low viscosity HPMC, and specifically the Methocel E5, is preferred. However, practically any hydrophilic polymer having a binding property (PVP, starch, hydrophilic cellulose derivatives, hydrophilic acrylate or methacrylate polymers) may be used. The drug layer typically constitutes 3-5% of the total weight of the bead composition (preferably about 4%). In relative amounts, the layer may typically comprise about 50-60% of tolterodine tartrate and 40-50% of the binder, typically HPMC, in respect to the mass of the whole drug layer.

The control release layer or membrane layer generally comprises a water insoluble (or pH-dependent soluble) polymer together with a pH-independent soluble polymer as a pore forming agent. Optionally, the mixture of polymers may be combined with a plasticizer, as is known in the art. The water insoluble polymer is typically ethyl cellulose. Among commercial brands of ethyl cellulose, the Surelease® brand is preferred. Surelease is available as an aqueous dispersion containing about 25% of the solid material, consisting primarily of ethyl cellulose plasticised with a fractionated coconut oil. The pore forming agent is typically hydroxypropylmethyl cellulose. Among commercial brands thereof, Methocel E5 is preferred. The release control layer typically constitutes 10-20 wt % of the total weight of the bead composition (preferably about 14 wt %). In relative amounts, the water insoluble polymer comprises about 80-90 wt % of the layer mass and the pore forming component comprises 10-20 wt %, preferably about 14 wt % of the layer mass.

In terms of relative weight ratios amongst the layers, the sealcoat layer typically comprises 25-30%, the drug layer 13-20% and the control release layer 50-65% of the overall weight of all three layers within the bead.

The beads of the present invention are controlled release beads, meaning in a broad sense that immediate release of the tolterodine has been disrupted. However, preferred embodiments of the invention exhibit the following release rate of tolterodine in a phosphate buffer pH 6.8: not more than 30% (preferably 5-25%) at 1 hour, from 40 to 85% (preferably 45-65%) at 3 hours and not less than 80% at 7 hours. A release rate that is relatively zero order is also preferred. In particular, it is desirable and commercially advantageous to have a dissolution profile that is equivalent to the current controlled release capsules of tolterodine, e.g. DETROL LA™ and/or DETRUSITOL SR™.

The process for producing the beads of the present invention can be carried out by any conventional or suitable techniques and typically comprises the following steps:

a) providing a core unit,

b) applying the first layer comprising the water soluble polymer on the core unit,

c) applying the second layer comprising the drug and the binder on the first layer, and

d) applying the third layer comprising the mixture of the water insoluble and water soluble polymer on the second layer.

Conventionally, the layering may be performed in fluid bed coating equipment, wherein the layers are applied stepwise on the material to be coated. The layering or coating operations are preferably performed by spraying a solution or dispersion of the respective layer materials onto the particle to be coated. The liquid vehiculum (liquid vehicle or carrier) is typically water. Similarly, any of the coating techniques described in the above-mentioned patents may also be applied. After any particular coating, the coated material may be dried, typically gently, before the application of the next coat. It should be taken in consideration that the yield and efficacy of the coating is never 100%. In particular, the considerable abrasion of the sugar sphere surface during the first coating has to be taken into account. Accordingly, corresponding adjustments in the percentage weights of the coating components may have to be performed.

Optionally, the bead disclosed above may also comprise a fourth, outermost layer for improving the mechanical properties of the produced pellet. Preferably, such layer comprises HPMC, and its relative amount is about 0.5-2%, preferably about 1% of the total weight of the bead. Such a layer is generally not a functional layer, i.e. it does not substantially modify the release rate. The layer is applied onto the bead in essentially the same manner as disclosed above.

After the final coating step, the beads are generally “cured”, usually in the same fluid bed system or in a tray drier system, by heating to a temperature of about 40-80° C., for 1-72 hours. Typically, the curing conditions are 50° C. and 8-48 hours.

The produced beads may be then formulated into a pharmaceutical formulation for the final application. Typically, such a formulation is represented by a capsule filled with a predetermined amount of the beads of the present invention. The capsule typically contains 50 to 250 mg of the beads, which comprises from 1 to 10 mg, preferably 2 mg or 4 mg, of tolterodine (as tartrate) per capsule. The formulation is administered orally.

### EXAMPLE 1

A controlled release bead having the following manufacturing formula was prepared

| | | |
|---|---|---|
| a) Core | sugar sphere | 74.98% |
| b) Sealcoat | HPMC | 7.0% |
| c) Drug layer | tolterodine tartrate | 2.22% |
| | HPMC | 1.80% |
| d) CR layer | ethylcellulose | 12.04% |
| | HPMC | 1.96% (14% of the CR coat) |

Corresponding amounts of the ingredients were weighed in respect to 65 kg of sugar cores used as a starting material. After analyzing the product after each coating step and taking in consideration the yield and the efficiency of the coating (74% of the first coating, 96% of the second, 89% of the third), the weighing of the ingredients were adjusted accordingly.

a) Preparation of Coating Materials

Coating Composition 1:

A 10% dispersion of HPMC Pharmacoat 603 (3 cP) in water was prepared and equilibrated for 24 hours under stirring. Dispersion was de-aerated before use.

Coating Composition 2:

A 3.5% dispersion of HPMC Methocel E5 (5 cP) in water was prepared and equilibrated for 24 hours under stirring. Calculated amount of tolterodine tartrate was added under gentle stirring and the mixture was homogenized by using ultrathurrax for 20-30 minutes. Dispersion was de-aerated before use.

Coating Composition 3:

A 6.1% dispersion of HPMC Methocel E5 (5 cP) in water was prepared and equilibrated for 24 hours under stirring. Calculated amount of Surelease (25% aqueous suspension of ethyl cellulose) was added under gentle stirring and mixed for 30 minutes. Dispersion was deaerated before use.

b) Process:

65 kg of sugar spheres (710-850 microns) were charged into the fluid bed coating equipment [Aeromatic Fielder MP4/5] preheated to 60° C. The spheres were heated at the same inlet air temperature for 5 minutes. The coating composition 1 was loaded through the spraying nozzle at the rate 352 g/min and an inlet temperature of 92° C. Air flow rate was 1200 m³/hour, nominal product temperature was 42° C. After a few minutes of drying, the product was analysed on yield and efficacy of coating. The coating composition 2 was applied at a spray rate of 365 g/min and an inlet temperature of 75° C., airflow rate was 1300 m³/hour, product temperature 32° C. After coating, the spheres were dried for a few minutes and analysed again. Finally, the coating composition 3 was applied at spray rate 270 g/min and an inlet temperature 75 C, airflow rate was 1300 m³/hour, product temperature 43 C. After ending the spraying, the nozzle(s) and tubing were flushed with water and the spheres were heated for a few minutes more. The coated beads were cured in the oven at 50° C. for 48 hours.

The prepared beads had the following final composition:

| | | |
|---|---|---|
| a) Core | sugar sphere | 77.92% |
| b) Seal coat | HPMC | 5.37% |
| c) Drug coat | Tolterodine tartrate | 2.21% |
| | HPMC | 1.79% |
| d) CR coat | ethylcellulose | 10.93% |
| | HPMC | 1.78% |

The spheres are filed into hard gelatine capsules. Filing weight is 90 mg (for the tolterodine tartrate 2 mg product) or 180 mg (for tolterodine tartrate 4 mg product).

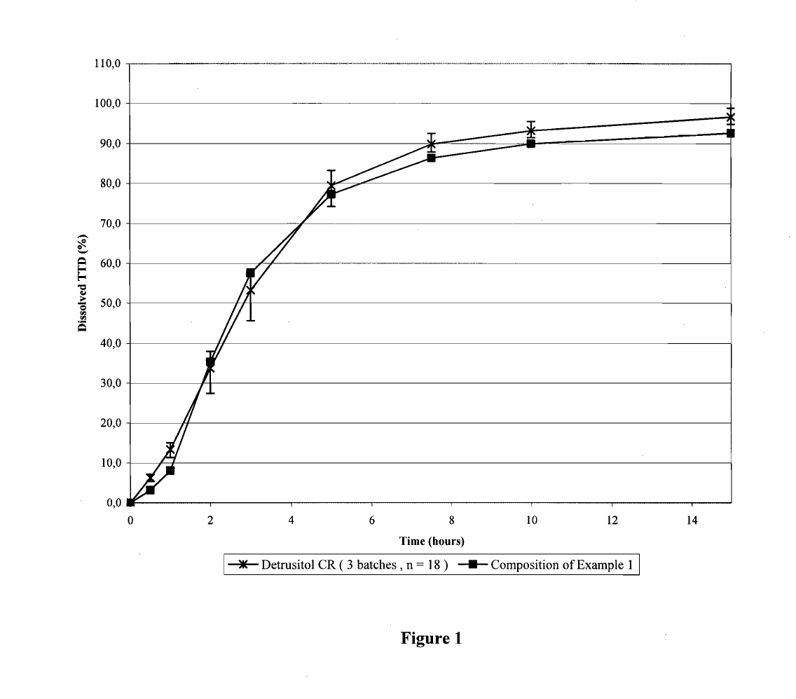

The release rate of the product is shown in FIG. 1 along with a 3 batch average (n=18) dissolution of DETRUSITOL SR™. The release was measured at 37° C. in 800 ml of USP phosphate buffer pH 6.8 on a USP dissolution test apparatus 1 at 100 rpm. The data in FIG. 1 were obtained by an HPLC method and are uncorrected. The results indicate that Example 1 has a favorable and/or equivalent dissolution curve to DETRUSITOL SR™.

Each of the patents and patent applications mentioned above are incorporated herein by reference. The invention having been described it will be obvious that the same may be varied in many ways and all such modifications are contemplated as being within the scope of the invention as defined by the following claims.

I claim

1. A controlled release pharmaceutical beads comprising tolterodine or an acid addition salt thereof as the active substance, comprising:
a) a sugar core having a diameter of 500-1000 microns, which constitutes 70 to 80% of the total weight of the bead composition;
b) a sealcoat layer surrounding said core, which constitutes 5-15% of the total weight of the bead composition, comprising hydroxypropylmethyl cellulose;
c) a drug layer, which constitutes 3-5% of the total weight of the bead composition, comprising said tolterodine or acid addition salt thereof and a hydrophilic polymer; and
d) a control release layer, which constitutes 10-20% of the total weight of the bead composition, comprising a water insoluble polymer and a pore forming component material.

2. The bead according to claim 1, wherein the diameter of the sugar core is from 700 to 850 microns.

3. The bead according to claim 1, wherein the mass of the sealcoat is from 5 to 10%.

4. The bead according to claim 1, wherein the tolterodine is tolterodine tartrate.

5. The bead according to claim 1, wherein the hydrophilic polymer in said drug layer is hydroxypropylmethylcellulose.

6. The bead according to claim 5, wherein the amount of the tolterodine tartrate is 50-60%, relative to the drug layer mass.

7. The bead according to claim 6, wherein the amount of the hydroxypropylmethyl cellulose is from 40 to 50% of the drug layer mass.

8. The bead according to claim 1, wherein the water insoluble polymer in said control release layer is ethyl cellulose.

9. The bead according to claim 8, wherein the pore forming component material is hydroxypropylmethyl cellulose.

10. The bead according to claim 8, wherein the water insoluble polymer comprises 80-90% of the weight of the control release layer.

11. The bead according to claim 9, wherein the hydroxypropylmethylcellulose comprises 10-20% of the weight of the control release layer.

12. The bead according to claim 1, which further comprises an outermost filmcoat.

13. The bead according to claim 12, wherein the filmcoat material comprises hydroxypropyl methylcellulose.

14. The bead according to claim 12, wherein the mass of the filmcoat is 0.5-2% of the weight of the bead.

15. The bead according to claim 1, wherein said bead exhibits the following release rate of tolterodine in a phosphate buffer pH 6.8: not more than 30% at 1 hour, from 40 to 85% at 3 hours and not less than 80% at 7 hours.

16. The bead according to claim 15, wherein 45-65% of tolterodine has been released at 3 hours.

17. A pharmaceutical composition, comprising a plurality of said controlled release beads of claim 1 in a capsule.
